(19) United States
(12) Patent Application Publication
(10) Pub. No.: US 2022/0096777 A1
(43) Pub. Date: Mar. 31, 2022

(54) BREATH SENSING WITH REMOTE PRESSURE SENSOR

(71) Applicant: Vapotherm, Inc., Exeter, NH (US)

(72) Inventor: Scott A. Leonard, Bedford, NH (US)

(21) Appl. No.: 17/484,374

(22) Filed: Sep. 24, 2021

Related U.S. Application Data
(60) Provisional application No. 63/083,388, filed on Sep. 25, 2020.

(51) Int. Cl. A61M 16/08; G01K 13/00; A61M 16/06; A61M 16/00

(52) U.S. Cl. CPC A61M 16/0858; G01K 13/00; A61M 2230/50; A61M 16/024; A61M 2016/0027; A61M 16/0666

(57) ABSTRACT

Devices and methods are provided herein for sensing and monitoring of patient breathing during high flow respiratory therapy. A nasal cannula having a sensing lumen and remotely-located pressure sensor is provided herein for breath sensing that provides an accurate and clear signal while high flow rates of breathing gas are delivered from the nasal cannula. Condensation can be removed from the sensing lumen by applying a constant, pulsatile, or burst flow of purge gas. The breath sensing apparatus can be integrated in a respiratory system with a breathing gas source or can be packaged as a separate device capable of adapting to a variety of systems.

### CROSS-REFERENCE TO RELATED APPLICATIONS

This application claims priority to and the benefit of U.S. Provisional Patent Application No. 63/083,388, filed on Sep. 25, 2020, and entitled “BREATH SENSING WITH REMOTE PRESSURE SENSOR”, the entire contents of the which is incorporated herein by reference.

### BACKGROUND

Patients with respiratory ailments are often treated with respiratory assist devices that deliver supplemental breathing gas to the patient. Such devices may deliver gas to the patient using high flow therapy (HFT). HFT devices deliver breathing gas at a high flow rate via an interface, such as a nasal cannula, to increase the patient's fraction of inspired oxygen (FiO₂), decrease the patient's work of breathing, or accomplish both. Increasing FiO₂ or decreasing the work of breathing helps the patient recover from respiratory ailments, such as respiratory distress or bronchospasms. Some HFT devices heat and humidify the delivered breathing gas for medical reasons (e.g., to maintain the pliability of the tissues of surfactant-deficient patients, or to preserve mucosal integrity) or to reduce patient discomfort.

In order to make respiratory clinical decisions for a patient, a clinician needs as much information as possible about the patient's current and historical medical status. For example, there are several methods available for measuring breathing, but many do so indirectly by looking at changes to the patient's heart rate, blood, or muscle movement. Conventionally, breath sensing measurements are available for patients receiving respiratory therapies that use a sealed mask, such a CPAP, BiPAP, mechanical ventilator, or other therapies. With a mask, accurate sensing is straightforward, because the mask seal delimits a closed system. But with high flow and high velocity nasal cannula systems, these measurements become more challenging, as the patient's nares are open to the environment. The flow rate from the cannula may exceed the patient's breathing flow rate and obscure the signal of pressure or temperature. Due to the open nature of the cannula interface, the pressure changes are very small, for example, on the order of 5 to 50 Pa, which are difficult to detect. Additionally, issues related to patient comfort are more problematic in the open nasal cannula systems and in systems which provide humidified breathing gas.

### SUMMARY

Devices and methods are provided herein for sensing and monitoring of patient breathing during high flow respiratory therapy. A nasal cannula having a sensing lumen and pressure sensor is provided herein for breath sensing that provides an accurate and clear signal while high flow rates of breathing gas are delivered from the nasal cannula, for example, at a high exit velocity. A length of tubing may be used to consistently convey the pressure changes to the pressure sensor from near the nares. The pressure sensor may be located remotely, and the tubing may be used to convey the pressure change between the nares and the remotely positioned sensor. From the pressure measurements, certain characteristics of the patient, such as breathing pattern or efficiency of breathing, can be determined and used to track the clinical status of the patient or to adjust the therapy.

Directly measuring air flow of patient breathing is preferable relative to conventional methods that indirectly assess or detect patient breathing via other physiological indicators. By locating the pressure sensor away from the nares of the patient, for example, at the end of a small-bore sensing lumen, pressure changes in the lumen that result from patient inspiration and expiration are reduced to lower frequencies that are more easily detected by the sensor, avoiding significant signal noise due to gas turbulence. Furthermore, condensation can be removed from the sensing lumen by applying a constant, pulsatile, or burst flow of purge gas. This can be beneficial in systems where the tubing is prone to occlusion by condensation, especially when the therapy is humidified. Capillary action tends to draw water into the tubing where it can mask the small pressure changes and damage the sensor.

The breath sensing apparatus can be integrated in a respiratory system with a breathing gas source or can be packaged as a separate device capable of adapting to a variety of systems. Information about a patient's breathing can be useful in several ways. A high flow or high velocity nasal cannula therapy device could make use of this data to report the information, adjust therapy, or ensure proper use of the device.

In one aspect, provided herein is a nasal cannula for breath sensing of a patient being provided high flow respiratory therapy. The nasal cannula includes a cannula body having a first connector configured to receive a first flow of breathing gas and a second connector configured to receive a second flow of breathing gas; a first nasal prong configured to deliver the first flow of breathing gas to a nare of the patient; a second nasal prong configured to deliver the second flow of breathing gas to a nare of the patient; and a pressure sensor system positioned on the cannula, on or near one or more of the nasal prong exits such that, during patient inspiration and expiration, the pressure of gas flowing in and out of the nare can be detected. In some implementations, the pressure sensor system is configured with a first sensing lumen with a first sensing tip positioned along the first nasal prong, such that gas flows entering and exiting the nares flow over the first sensing tip to create a pressure change within the first sensing lumen. A first pressure sensor is also provided in the pressure sensing system, the sensor being configured to detect a first pressure in the first sensing lumen. For example, the pressure sensor may be positioned at a proximal end of the first sensing lumen opposite the first sensing tip. A cannula configured with such a pressure sensor system provides the advantage of deriving information about a patient's breathing while functioning with high velocity or high flow nasal cannula therapy. The remote positioning of the pressure sensor and use of a sensing lumen with distal tip allows for detection of pressure changes without substantial turbulence, noise, or high frequency pressure fluctuations at the pressure sensor, thus overcoming obstacles resulting from the open nature of nasal cannula therapy.

In some implementations, multiple pressure sensor systems may be used. For example, the nasal cannula may further include a second sensing lumen having a second sensing tip positioned along the second nasal prong such that, during patient inspiration and expiration, gas flows over the second sensing tip to create a second pressure change within the second sensing lumen; and a second pressure sensor configured to detect a second pressure in the second sensing lumen and positioned at a proximal end of the second sensing lumen opposite the second sensing tip. In some implementations, the first sensing lumen includes a housing mounted on the cannula body, and a tube configured to enable fluid communication between the first sensing tip and the pressure sensor, wherein the tube is in fluid communication with the pressure sensor at the proximal end of the first sensing lumen, and wherein the housing is fluidically connected to the tubing. The tube may have a length between about 5 mm and about 3 m and an internal diameter between about 0.25 mm and about 2.5 mm.

In some implementations, a control gas flows from a flow generator through at least a portion of the pressure sensing tube in a direction from the proximal end to the sensing tip. The control gas collides with patient breath at a boundary region adjacent to the first sensing tip. The control gas may be delivered continuously or intermittently as a bolus flow. In some implementations, the control gas has a flow rate between about 0.1 mL/min and about 10 mL/min. The flow generator may be one of a fan, a syringe pump, a blower, a compressor, a bellows, or a wall air source. In some implementations, the nasal cannula is configured with the control gas so as to purge condensation from one or more sensing lumens. In some implementations, a sensing lumen includes a tee joint positioned along the tube and configured to convey the control gas into the tube from the flow generator. In some implementations, control gas is provided for first and second sensing lumens. In some implementations, the nasal cannula further includes a temperature sensor configured to measure a breath temperature.

In some implementations, the detected pressure by the pressure sensing system indicates a difference between an internal pressure within the first sensing lumen and ambient pressure of the surrounding environment. The difference may be about 1 Pa to about 5 Pa. The difference may be at least about 1 Pa or at least about 5 Pa. In some implementations, the first pressure sensor is configured to output a pressure signal indicative of the detected first sensing lumen pressure. A second pressure sensor may be similarly configured.

In some implementations, the first sensing tip has an exit port that is angled relative to a longitudinal axis of the first sensing lumen. The exit port may include a tip face at an angle other than 90 degrees relative to the longitudinal axis of the sensing lumen. For example, the angle may be between about 30 degrees and about 60 degrees. In some implementations, the angle is about 45 degrees. In some implementations, the angle is about 30 degrees or about 60 degrees. In some implementations, the tip face has rounded, beveled, or chamfered edges. In some implementations, the sensing tip is positioned on an exterior surface of the first nasal prong. In some implementations, the sensing tip is positioned on a dorsal side of the exterior surface of the first nasal prong. In some implementations, the sensing tip is positioned proximally to a prong tip of the first nasal prong. In some implementations, the sensing tip is positioned on the nasal cannula such that the sensing tip resides within the nare of the patient while the cannula is in use. The sensing tip may have an internal diameter between about 0.25 mm and 2.5 mm. In some implementations, the first nasal prong has an internal diameter of about 1.0 mm to about 4.0 mm. For example, the ratio of sensing tip diameter to nasal prong diameter is between about 0.0625 and about 2.5.

In another aspect, provided herein is a breath sensing device configured to monitor patient breathing. The breath sensing device includes the nasal cannula of any of the above implementations of the above aspect and a controller configured to receive a pressure signal from the first pressure sensor indicative of the first sensing lumen pressure and convert the pressure signal into data. In addition to the advantages of the nasal cannula recited above, the breath sensing device is advantageous as an adaptable device that can provide breath sensing and monitoring capabilities to existing respiratory therapy systems.

In some implementations, the device is configured to couple to a respiratory therapy system. The controller may be configured to send instructions to the respiratory therapy system to change an operating parameter of the respiratory therapy system, or the controller may be configured to send the data to the respiratory therapy system for determining a change to an operating parameter of the respiratory therapy system. The respiratory therapy system is configured to provide the first flow of breathing gas to the nasal cannula.

In some implementations, the controller is configured to determine periods of patient inspiration and/or expiration based on the data from the first pressure sensor. The controller may be configured to determine and display at least one of patient expiratory/inspiratory ratio, a current breath rate, or a patient breathing pattern based on the data from the first pressure sensor. In some implementations, the controller is configured to apply an algorithm to the data. In some implementations, the algorithm is configured to determine an average pressure in the data, determine maximum and minimum peaks in the data, filter the data, determine an average maximum peak over a period of time, determine an average minimum peak over a period of time, and/or determine a pressure threshold as a percentage of the average maximum peak. The algorithm may be configured to identify a breath when the pressure crosses from below the average pressure to above the average pressure, then exceeds the average pressure by at least the pressure threshold, then reaches a maximum peak, then crosses from above the average pressure to below the average pressure, then reaches a minimum peak, and then crosses from below the average pressure to above the average pressure. In some implementations, the algorithm is configured to identify and exclude non-breathing periods from the data.

In some implementations, the nasal cannula further includes a second sensing lumen having a second sensing tip positioned along the second nasal prong such that, during patient inspiration and expiration, gas flows over the second sensing tip to create a second pressure change within the second sensing lumen. In some implementations, the nasal cannula further includes a second pressure sensor configured to detect a second pressure in the second sensing lumen and positioned at a proximal end of the second sensing lumen opposite the second sensing tip. The controller may be configured to average data from the first pressure sensor and the second pressure sensor to generate an average sensing lumen pressure. The controller may be configured to receive data from only one of the first pressure sensor or the second pressure sensor when one of the nares of the patient is blocked.

In another aspect, provided herein is a respiratory therapy system. The respiratory therapy system includes the nasal cannula of any of the above implementations of the above aspect, a capital unit including a pressurized gas source, and a delivery tube configured to convey the first flow of breathing gas from the capital unit to the first connector of the nasal cannula. The respiratory therapy system may further include a controller configured to receive a pressure signal from the first pressure sensor indicative of the first sensing lumen pressure and convert the pressure signal to data. In addition to the advantages of the nasal cannula recited above, the respiratory therapy system is advantageous by allowing for feedback control of the respiratory therapy provided by the capital unit based on the information derived from the breath sensing data.

In some implementations, the controller is configured to change an operating parameter of the capital unit based on the received data. In some implementations, the controller is configured to determine periods of patient inspiration and/or expiration based on the data from the first pressure sensor. In some implementations, the controller is configured to determine and display a patient expiratory/inspiratory ratio, current breath rate, and/or a patient breathing pattern based on the data from the first pressure sensor.

In some implementations, the controller is configured to apply an algorithm to the data. In some implementations, the algorithm is configured to determine an average pressure in the data, determine maximum and minimum peaks in the data, filter the data, determine an average maximum peak over a period of time, determine an average minimum peak over a period of time, and/or determine a pressure threshold as a percentage of the average maximum peak. The algorithm may be configured to identify a breath when the pressure crosses from below the average pressure to above the average pressure, then exceeds the average pressure by at least the pressure threshold, then reaches a maximum peak, then crosses from above the average pressure to below the average pressure, then reaches a minimum peak, and then crosses from below the average pressure to above the average pressure. In some implementations, the algorithm is configured to identify and exclude non-breathing periods from the data.

In some implementations, the nasal cannula further includes a second sensing lumen having a second sensing tip positioned along the second nasal prong such that, during patient inspiration and expiration, gas flows over the second sensing tip to create a second pressure change within the second sensing lumen. In some implementations, the nasal cannula further includes a second pressure sensor configured to detect a second pressure in the second sensing lumen and positioned at a proximal end of the second sensing lumen opposite the second sensing tip. The controller may be configured to average data from the first pressure sensor and the second pressure sensor to generate an average sensing lumen pressure. The controller may be configured to receive data from only one of the first pressure sensor or the second pressure sensor when one of the nares of the patient is blocked.

In any of the above aspects, the first flow of breathing gas may be heated and humidified (e.g., by a vapor transfer unit or a hotpot humidifier). The first flow of breathing gas may be configured to exit the first nasal prong at a high velocity, for example, at an exit velocity of at least about 40 m/s and/or to be delivered at a flow rate between about 1 L/min and 60 L/min.

In another aspect, provided herein is a method for sensing patient breathing when the patient is provided with high flow respiratory therapy. The method includes the steps of delivering a flow of breathing gas to a nare of the patient via a nasal cannula; and detecting a pressure in a sensing lumen of the nasal cannula using a pressure sensor positioned at a proximal end of the sensing lumen opposite a tip of the sensing lumen. The sensing lumen is positioned such that, during patient inspiration and expiration, gas flows over the tip of the sensing lumen to create a pressure change within the sensing lumen. The remote positioning of the pressure sensor and use of a sensing lumen with distal tip allows for detection of pressure changes without substantial turbulence, noise, or high frequency pressure fluctuations at the pressure sensor, thus overcoming obstacles resulting from the open nature of nasal cannula therapy.

In some implementations, the method further includes transmitting a signal indicative of the detected pressure from the pressure sensor to a controller. The method may further include processing the signal using an algorithm stored in a memory of the controller. In some implementations, processing the signal includes determining a current breath rate, and the method further includes displaying the current breath rate. The signal may be indicative of a plurality of pressures detected by the pressure sensor over a period of time, and processing the signal includes identifying peaks in the plurality of detected pressures over time. The method may further include determining an increase or decrease in pressure over time. The method may further include identifying an apnea event based on the signal and logging the apnea event.

The method may further include determining an average pressure in the data. The method may further include determining maximum and minimum peaks in the data and/or filter the data. The method may further include determining an average maximum peak over a period of time. The method may further include determining an average minimum peak over a period of time. The method may further include determining a pressure threshold as a percentage of the average maximum peak. The method may further include determining a breath when the pressure crosses from below the average pressure to above the average pressure, then exceeds the average pressure by at least the pressure threshold reaches a maximum peak, then crosses from above the average pressure to below the average pressure, then reaches a minimum peak, and then crosses from below the average pressure to above the average pressure.

In some implementations, the method further includes operating the controller to alter an operating parameter of the respiratory therapy based on the detected pressure. The operating parameter may be at least one of a breathing gas flow rate, an oxygen percentage of the flow of breathing gas, a breathing gas temperature, a breathing gas humidity, or an aerosolized medicament flow rate. In some implementations, the detected pressure is a differential pressure between a pressure within the sensing lumen and an ambient pressure. In some implementations, the method further includes conveying a control flow of gas through the sensing lumen from a flow generator. The method may further include purging condensation from the sensing lumen with the control flow of gas. In some implementations, the method further includes humidifying the flow of breathing gas using a vapor transfer unit or a hotpot humidifier.

In another aspect, provided herein is a method of manufacturing a nasal cannula for respiratory therapy and breath sensing. The method includes the step of maintaining a first mandrel, a second mandrel, and a third mandrel in a fixed arrangement with respect to each other, wherein the third mandrel is positioned along at least a portion of the first mandrel at a distance from the first mandrel; coating the arrangement with a material, The method includes the step of curing the coated arrangement; trimming at least one coated mandrel to create an opening in the coating of the trimmed mandrel. The method includes the step of removing the cured coating from the arrangement, wherein the coating on the first, second, and third mandrels form a first nasal prong, a second nasal prong, and a sensing lumen, respectively.

In some implementations, the step of coating includes immersing the arrangement in the material and removing the arrangement from the material. In some implementations, the step of coating includes spraying the material onto the arrangement. In some implementations, the first, second, and third mandrels are fixedly held on a substrate.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1 shows a nasal cannula with a breath sensing lumen, according to an illustrative implementation;

FIG. 2 shows a flowchart outlining a method for breath sensing during respiratory therapy, according to an illustrative implementation;

FIG. 3 shows a flowchart outlining a method of manufacturing of a breath sensing nasal cannula, according to an illustrative implementation; and

FIG. 4 shows a block diagram of a respiratory therapy system configured to monitor patient breathing, according to an illustrative implementation.

### DETAILED DESCRIPTION

To provide an overall understanding of the assemblies and methods described herein, certain illustrative implementations will be described. These illustrative implementations are described for high velocity respiratory therapy, which can be understood to mean that breathing gas is delivered to the patient at a velocity of at least about 40 m/s, for example, exiting from a nasal cannula prong. Although the implementations and features described herein are specifically described for high velocity respiratory therapy, it will be understood that all the components and other features outlined below may be combined with one another in any suitable manner and may be adapted and applied to other respiratory therapy systems and devices, including low flow oxygen therapy, continuous positive airway pressure therapy (CPAP), mechanical ventilation, oxygen masks, Venturi masks, or Tracheostomy masks, or combinations thereof. Although the features are described within regard to over-ear lariat-type nasal cannula securement systems, it will be understood that the components and other features outlined below can be applied to other nasal cannula (or tracheostomy mask) securement systems, including head gear, adhesive-based systems, or braces. The term “about,” as used herein, should be understood to mean plus or minus 20%. For example, “about 20 mm” should be understood to mean 20 mm±4 mm.

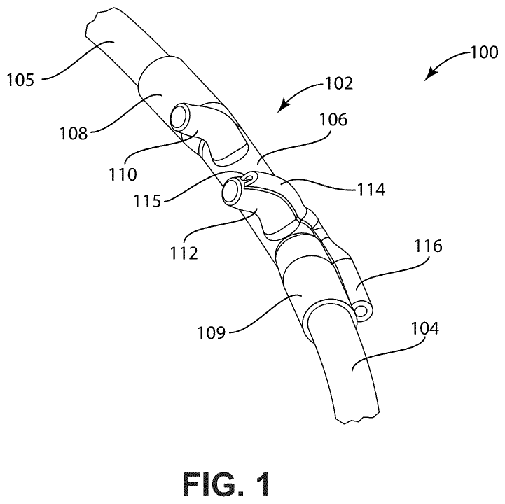

FIG. 1 shows a system 100 including a nasal cannula 102 and breathing gas supply tubes 104 and 105. Nasal cannula 102 includes a cannula body 106 having connectors 108 and 109 adapted to couple to breathing gas supply tubes 104 and 105, respectively, to receive flows of breathing gas. Nasal cannula 102 further includes nasal prongs 110 and 112 adapted to convey the flows of breathing gas into the nares of a patient. A sensing lumen 114 is disposed along nasal prong 112. Sensing lumen 114 includes a tip 115 and a housing 116 adapted to couple to a tube (not shown). Tip 115 may be disposed at a distal end of sensing lumen 114. As breathing gas is delivered to the patient via prongs 110 and 112, inhaled or exhaled gas flows past tip 115 or into sensing lumen 114 and changes the pressure within sensing lumen 114. A pressure sensor (not shown) may be disposed at a proximal end of sensing lumen 114 and detect the pressure changes within sensing lumen 114.

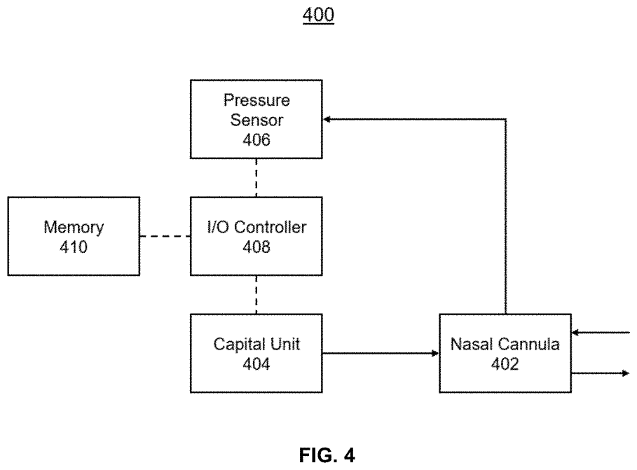

The pressure sensor is configured to output a pressure signal indicative of the detected pressure. By locating the pressure sensor away from cannula and, for example, at the end of the tube extending from the proximal end of sensing lumen 114, the pressure at the pressure sensor is conditioned to reduce noise in the signal. The pressure signal outputted by the pressure sensor may be communicated to a controller, for example, as described below in relation to FIG. 4. A pressure sensor capable of resolving pressure differences as small as about 1 Pa to about 4 Pa may be suitable. Such a high sensitivity pressure sensor allows detection of very small changes in pressure, for example, changes of about 1 Pa to about 4 Pa, or changes of less than 1 Pa. The pressure sensor may be configured to measure or determine absolute pressure within sensing lumen 114 or to measure or determine differential pressure relative to atmospheric pressure of the surrounding environment. In the latter implementation, the pressure sensor has a high resolution and includes an additional probe configured to measure the atmospheric pressure of the surrounding environment.

When the patient breathes, air flows past tip 115. Depending on whether the patient has their mouth open or closed and what the therapy flow rate is, the direction of flow past tip 115 may reverse during each breath or may only increase and decrease while maintaining the same direction. Either event causes pressure changes within sensing lumen 114, which may be detected or measured using the pressure sensor. The pressure may always be less than the environmental pressure (typically the case with open mouth breathing where the flow does not reverse) or may swing from positive to negative (typical with closed mouth breathing). When a patient stops breathing or removes cannula 102, the pressure changes stop and may be detected as an absence of pressure change.

In some implementations, cannula 102 includes a second sensing lumen having a second sensing tip positioned along the second nasal prong such that, during patient inspiration and expiration, gas flows over the second sensing tip to create a second pressure change within the second sensing lumen. In some implementations, cannula 102 includes a second pressure sensor configured to detect a second sensing lumen pressure in the second sensing lumen and positioned at a proximal end of the second sensing lumen opposite the second sensing tip. When two sensing lumens and two pressure sensors are used, the two detected pressures may be averaged to generate an average sensing lumen pressure. Alternatively, one of the two pressure sensors may be chosen, for example, using a controller as described herein and configured to receive a pressure signal from each pressure sensor, for example, if one nare becomes occluded such that inhaled or exhaled gas only or mostly flows over one of the two sensing tips.

Housing 116 is mounted on cannula body 106. For example, housing 116 may be integrally formed with cannula body 106. Alternatively, housing 116 may be detachably affixed to cannula body 106 via a clip-on connector, a magnet, a sliding lock, or another suitable form of attachment. A tube may be attached to the housing 116, for example at a proximal end of housing 116. The tube enables fluid communication between tip 115 and the pressure sensor. The tube and housing 116 define a fixed volume between tip 115 and the pressure sensor, and the pressure within this fixed volume changes as inhaled or exhaled gas flows toward tip 115 from the nare. The fixed volume is partially determined by the tube length and the tube inner diameter. Microbore tubing may be used such that the inner diameter is small, e.g., about 0.25 mm to about 2.5 mm. The small inner diameter minimizes the volume of air flow needed to conduct the pressure change along to the pressure sensor at the proximal end (of the tube). A flexible tube may be used in order to facilitate movement and repositioning of the pressure sensor while cannula 102 is in use. The tube may have a length between about 5 mm and about 3 m. As discussed above, the length of tube can serve to condition the pressure signal by reducing high frequency pressure fluctuations.

Tip 115 may be positioned on an exterior surface of prong 112. For example, tip 115 is positioned on a dorsal side of prong 112, for example, as shown in FIG. 1. For example, tip 115 may be positioned on a side of prong 112 farthest from the patient or on a top side of prong 112. Alternatively, tip 115 may be spaced from the tip of prong 112. Longitudinally, tip 115 may be flush with the tip of prong 112 or located proximally relative to the tip of prong 112, such that prong 112 extends farther into the nare than tip 115. In some implementations, tip 115 is set back from the tip of prong 112 such that tip 115 is outside of the nare while prong 112 extends at least partially into the nare.

Tip 115 may be angled relative to a longitudinal axis of sensing lumen 114. As noted previously, breath sensing with a high flow nasal cannula can be particularly difficult due to the high flow rates and open nature of the system, leading to high noise in the pressure signal. Constructing sensing lumen 114 with an angled tip 115 may reduce turbulence of the inhaled and/or exhaled gas, improving the pressure signal. For example, tip 115 may include a tip face at an angle other than 90 degrees relative to the longitudinal axis, such that the tip face is angled away from the outlet of prong 112. In some implementations, the angle is between about 30 degrees and about 60 degrees. In some implementations, the angle is between about 5 degrees and about 85 degrees. In some implementations, the angle is about 45 degrees. In some implementations, the angle is about 30 degrees or about 60 degrees. In some implementations, the angle is about 5 degrees or about 85 degrees. The tip face may have rounded, beveled, or chamfered edges, for example, to further reduce turbulence and prevent irritation or discomfort to the patient. Tip 115 may have an internal diameter between about 0.25 mm and about 2.5 mm. Tip 115 may have an internal diameter between about 0.10 mm and about 4.00 mm. In some implementations, tip 115 may have an internal diameter of about 0.25 mm or about 2.5 mm. In some implementations, tip 115 may have an internal diameter of about 0.5 mm, about 1.0 mm, about 1.5 mm, about 2.0 mm, or about 2.5 mm.

In some implementations, a control gas flows from a flow generator through at least a portion of the tube and/or sensing lumen 114. The control gas flows in a direction from the proximal end (e.g., of the tube) toward tip 115. In some implementations, the control gas flows in a direction from the proximal end of the sensing lumen 114 toward tip 115. The control gas may be introduced into the tube at a point near the pressure sensor, for example, via a tee joint in the tube that is fluidically connected to the flow generator. The control gas prevents liquids from entering sensing lumen 114 or the tube. The flow of control gas may be continuous or intermittent (e.g., as a pulse or single bolus of flow). A continuous flowrate of about 0.1 mL/min to about 10 mL/min can be effective to both prevent liquid ingress and maintain a stable, low pressure at the pressure sensor. The control gas may be supplied by a low pressure source (e.g., at <275 kPa, <200 kPa, <150 kPa, <100 kPa, <50 kPa, <30 kPa, <20 kPa, <10 kPa, <5 kPa or any other suitable gauge pressure). For an intermittent pulse or single bolus, a volume of about 1 mL to about 5 mL can clear liquid near tip 115 or elsewhere in sensing lumen 114. The control gas exits sensing lumen 114 at tip 115 and collides with inhaled or exhaled gas at a boundary region adjacent to tip 115, such that changes in pressure due to patient breathing are still detectable by the pressure sensor via sensing lumen 114. Suitable flow generators include a fan, a syringe pump, a blower, a compressor, a bellows, a wall air source, a pressurized tank, a piezo pump, or any other suitable source of pressurized gas. The flow generator may be incorporated in a respiratory system in use with cannula system 100 or may be separately configured with system 100 in a breath sensing device configured to adapt to various respiratory systems. For certain flowrates of control gas that lead to a high pressure at the pressure sensor, a pressure sensor with a larger pressure range may be used.

Suitable sources of pressurized breathing gas will be known to one of ordinary skill in the art. For example, the source may be the Vapotherm Flowrest System, Vapotherm HVT2.0 System, Precision Flow unit, or the Vapotherm 2000i, all of which are provided by Vapotherm, Inc. of Exeter, N.H., USA. For example, the source may be the respiratory therapy systems described in U.S. patent application Ser. No. 16/901,902 and U.S. patent application Ser. No. 15/783,566 (U.S. Publication No. 2018/0104436), each of which is incorporated herein by reference in its entirety. Other suitable sources of breathing gas will be known to one of ordinary skill in the art from the description herein, such as a blower, a compressor, a pressurized tank, a wall air outlet, or any other suitable gas source capable of generating high flow rates.

The source of breathing gas may be fluidically connected to first and second gas supply tubes 104 and 105 via a delivery tube (not shown) including an inlet configured to receive breathing gas from the source and a split outlet configured to divide and transmit breathing gas into the flows of breathing gas to the first and second gas supply tubes 104 and 105, respectively. The delivery tube may be insulated by an air or water jacket, or may be heated by an internal heated wire so as to maintain/regulate a temperature of the breathing gas provided to the patient. The tube providing the pressure to the pressure sensor is maintained separately from the delivery tube and external to any insulating means. In some implementations, the sensing lumen 114, tube, and/or pressure sensor are integrated into the delivery tube. For example, the tube conveying the pressure to the pressure sensor may be enclosed within a lumen of the delivery tube or embedded in a wall of the delivery tube, or the pressure sensor may be embedded in or affixed to the wall. The delivery tube may also serve to enclose any wires needed to connect the pressure sensor to a controller as described herein or the respiratory therapy system. The system 100 and delivery tube can be provided as a single unit to be attached to a variety of respiratory systems.

System 100 may be integrated in a respiratory therapy system having a breathing gas source and, optionally, a humidifying device (e.g., a vapor transfer unit or hotpot humidifier). Alternatively, system 100 may be implemented as an add-on unit configured to couple to a compatible respiratory therapy system with a breathing gas source and, optionally, a humidifying device (e.g., a vapor transfer unit or hotpot humidifier). System 100 may be used with a system that provides heated and humidified breathing gas. System 100 may be integrated into system 400 of FIG. 4 as nasal cannula 402, described below.

Suitable flowrates of breathing gas range from about 3 L/min to about 80 L/min. In some implementations, the flowrates of breathing gas is between about 20 L/min and about 70 L/min. In some implementations, the flowrates of breathing gas is between about 30 L/min and about 60 L/min. In some implementations, the breathing gas flowrate and nasal prong inner diameter are chosen such that the exit velocity of breathing gas from nasal prongs 110 and 112 is at a high velocity, for example, at least about 40 m/s. In some implementations, the exit velocity is between about 40 m/s and about 75 m/s for a balance effective flushing of CO₂ from the patient airway and patient comfort or noise reduction. In some implementations, an exit velocity between about 50 m/s and about 60 m/s is used. In some implementations, nasal prongs 110 and 112 have an internal diameter of about 1.0 mm to about 4.0 mm. In some implementations, nasal prongs 110 and 112 may have an internal diameter of about 1.5 mm to about 3.5 mm. In some implementations, nasal prongs 110 and 112 may have an internal diameter of about 2.0 mm to about 3.0 mm. For example, the ratio of sensing tip diameter to nasal prong diameter is between about 0.0625 and about 2.5.

In some implementations, a temperature sensor is used in addition to or in place of the pressure sensor. A temperature sensor can be applied in this context by measuring changes in temperature of the air near tip 115, because exhaled gas will have a higher temperature coming from within the patient's body than inhaled air from the atmosphere, presumed to be at or around room temperature, or possibly higher than room temperature due to heating of the delivered breathing gas. When using a temperature sensing in conjunction with the pressure sensor, the temperature sensing reading may be used to verify correlation between pressure signals and breathing patterns.

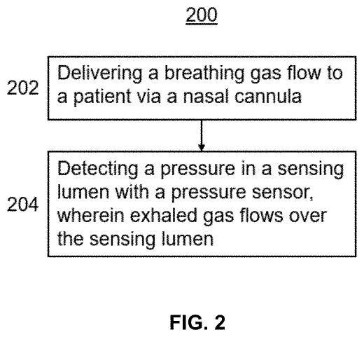

FIG. 2 shows a flowchart outlining an example method 200 for breath sensing while providing respiratory therapy to a patient. Method 200 includes steps 202 and 204. Step 202 involves delivering a flow of breathing gas to a nare of the patient via a nasal cannula. The nasal cannula includes a sensing lumen in fluid communication with a pressure sensor. Step 204 involves detecting a pressure within the sensing lumen of the nasal cannula using the pressure sensor. The sensing lumen is positioned such that gas inhaled or exhaled by the patient flows over a tip of the sensing lumen while the cannula is in use. The cannula may be cannula 102 described in relation to FIG. 1.

Method 200 may further include transmitting a signal indicative of the detected pressure from the pressure sensor to a controller, such as controller 408 of FIG. 4. The signal may indicate a detected pressure at a time point or a plurality of detected pressures over a period of time. The controller may be or may include a processor. The controller may be operatively coupled to a memory configured to store the signal, data based on the signal, and/or one or more algorithms for processing the signal. Method 200 may further include processing the signal using an algorithm stored in the memory. In some implementations, processing the signal includes determining a current breath rate or inspiratory/expiratory ratio of the patient. In some implementations, processing the signal includes identifying peaks in detected pressures over time. In some implementations, processing the signal includes determining an increase or decrease in pressure over time. In some implementations, processing the signal includes identifying an apnea event or a breathing pattern. In some implementations, processing the signal includes filtering, averaging, or smoothing the pressure data.

In some implementations, the algorithm is configured to determine an average pressure in the signal/data. The algorithm may determine maximum and minimum peaks in the data. The algorithm may determine the average maximum peak and/or the average minimum peak over a period of time. A pressure threshold may be determined as a percentage (e.g., between about 10% and about 30%) of the average maximum peak. This may allow the algorithm to identify a single breath of the patient based on when the pressure signal follows the following pattern: crosses the average pressure from below the average to above the average, then exceeds the average by at least the pressure threshold, then reaches a maximum peak, then cross the average from above the average to below the average, then reaches a minimum peak, and then crosses the average from below the average to above the average. The above steps may be based on a positive pressure swing, meaning that the pressure change is positive when the patient inhales. Alternatively, the pressure threshold may be determined as a percentage of the average minimum peak, and the algorithm steps are based on a negative pressure swing such that the pressure change is negative when the patient inhales. The pressure sensor used herein can be a relative pressure sensor, measuring the difference in pressure between two ports with one port positioned within the sensing lumen and one outside (measuring pressure of the surrounding environment), so the ports can be swapped to switch between the bases of positive and negative pressure swings.

Data representing information such as pressure signal, current breath rate, inspiratory/expiratory ratio, identified peaks, identified apnea events, or breathing patterns may be stored in the memory, for example, as an additional method step. The method may further include generating for display any of the above-listed information.

The above data and/or signal can be used to inform users, for example, clinicians, particularly as support for making clinical decisions related to respiratory therapy. For example, any of the above data can be displayed for informing a user or clinician. The data can also be transmitted to the gas source or another device to inform feedback-based control decisions. For example, information like breathing pattern or inspiratory/expiratory ratio determined from the pressure signal can be used to make adjustments to the provided therapy in a control loop or determine recommendations for therapy or treatment. Adjustments may include changing the breathing gas flowrate, breathing gas humidity, breathing gas temperature, oxygen percentage, oxygen flow rate, or aerosol concentration. These functionalities are described further in relation to FIG. 4 below.

In some implementations, the method includes conveying a control gas through the sensing lumen from a flow generator. As discussed above, the control gas prevents liquids from entering the sensing lumen. This feature is particularly helpful when the breathing gas is humidified, which leads to a higher likelihood of condensation buildup at the cannula. The flow of control gas may be continuous or intermittent (e.g., as a pulse). The control gas may be introduced into the tube at a point near the pressure sensor, for example, via a tee joint in the tube that is fluidically connected to the flow generator. A continuous flowrate of about 0.1 mL/min to about 10 mL/min can be effective to both prevent liquid ingress and maintain a stable, low pressure at the pressure sensor. In some implementations, a continuous flowrate can be between about 1.0 mL/min and about 7.5 mL/min, or between about 2.0 mL/min and 5 mL/min. The control gas may be supplied by a low pressure source (e.g., at <275 kPa, <200 kPa, <150 kPa, <100 kPa, <50 kPa, <30 kPa, <20 kPa, <10 kPa, <5 kPa or any other suitable gauge pressure). For an intermittent pulse, a volume of about 1 mL to about 5 mL can clear liquid near the tip of the sensing lumen. The control gas exits the sensing lumen at the tip and collides with inhaled or exhaled gas at a boundary region adjacent to the tip, such that changes in pressure due to patient breathing are still detectable by the pressure sensor via the sensing lumen. Suitable flow generators include a fan, a syringe pump, a blower, a compressor, a bellows, a wall air source, a pressurized tank, or any other suitable source of pressurized gas. The flow generator may be incorporated in a respiratory system in use with the cannula or may be separately configured with the in a breath sensing device configured to adapt to various respiratory systems. For certain flowrates of control gas that lead to a high pressure at the pressure sensor, a pressure sensor with a larger pressure range may be used.

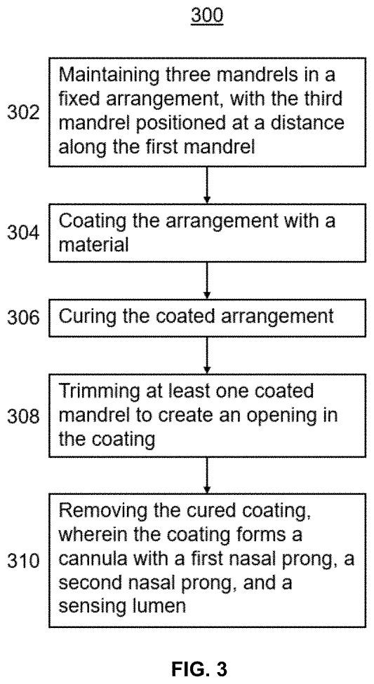

FIG. 3 shows a flowchart describing a method 300 for manufacturing a nasal cannula for respiratory therapy and breath sensing. Method 300 includes steps 302, 304, 306, 308, and 310. Step 302 involves maintaining a first mandrel, a second mandrel, and a third mandrel in a fixed arrangement with respect to each other. In the fixed arrangement, the third mandrel is positioned along at least a portion of the first mandrel at a distance from the first mandrel. Step 304 involves coating the arrangement with a material. Step 306 involves curing the coated arrangement. Step 308 involves trimming at least one coated mandrel to create an opening in the coating of the trimmed mandrel. Step 310 involves removing the cured coating from the arrangement. The coating on the first, second, and third mandrels form a first nasal prong, a second nasal prong, and a sensing lumen, respectively, of the nasal cannula. For example, the cured coating may be a flexible plastic that is in the shape of the nosepiece shown in FIG. 1.

In some implementations, in step 304, coating the arrangement includes immersing the arrangement in the material and removing the arrangement from the material (e.g., dip molding). The relatively low monetary cost of the dip molding material allows for discarding the excess material without significantly affecting the production cost. In some implementations, in step 304, coating the arrangement includes spraying the material onto the arrangement. In an example, the first, second, and third mandrels are fixedly held on a substrate. This may keep the mandrels in the desired arrangement for the step of coating. The third mandrel may be held at a distance from the first mandrel in the arrangement. All of the first, second, and third mandrels may be fixed in the arrangement with a spacing between each other. By leaving a small distance between the mandrels, the material that flows around the mandrels will separate the structures formed by the mandrels with a thin layer of material.

For example, the coated arrangement may be cured at room temperature, or at a temperature or set of temperature (such as those determined by the material's curing temperature profile) determined to accelerate the curing time. For example, the coating may be cured using a heat lamp, oven, UV radiation, or any other suitable means. After curing, at least one coated mandrel may be trimmed to create an opening in the coating of the trimmed mandrel.

The mandrels may be made of, for example, steel, aluminum-bronze alloys, stainless steel, or any other suitable material, such as those resistance to curing methods and which will not permanently adhere to the flexible material used in forming the nosepiece. The materials used to manufacture the cannula in general may also be selected so as to facilitate smooth flow within the tubing and appropriate durability and connectivity on the outside of the tubing. In some implementations a material of the outer surface includes at least one of: polyvinyl chloride (PVC) plastic, plastisol, vinyl, silicone, non-latex rubber, an elastomer, ethylene vinyl acetate (EVA), styrene-butadiene copolymer (SBC), polyether ether ketone (PEEK), a polyether block amide (such as PEBAX), a polyethylene material, a high-density polyethylene (HDPE) material, a medium-density polyethylene (MDPE) material, a low-density polyethylene (LDPE) material, a crack-resistant material, a material with a low coefficient of friction, a material less than 70 Durometer Shore A, and flexible plastic. Flexible plastics and the other material examples listed above may be chosen to provide customized comfort to a patient. For example, an infant patient may require a more flexible nosepiece than an adult patient. In such an example, a more flexible material may be chosen.

FIG. 4 shows a block diagram of a system 400 including nasal cannula 402, capital unit 404, pressure sensor 406, controller 408, and memory 410. Capital unit 404 includes a pressurized breathing gas source (not shown) configured to deliver a flow of breathing gas to nasal cannula 402. Nasal cannula 402 is configured to convey the flow of breathing gas at a high velocity (e.g., at least at about 40 m/s) into the nares of a patient. In some implementations, the exit velocity is between about 40 m/s and about 75 m/s. In some implementations, an exit velocity between about 50 m/s and about 60 m/s is used. As the patient breathes, a sensing lumen of nasal cannula 402 conveys pressure changes to pressure sensor 406, wherein the pressure changes result from patient inspiration and expiration. Pressure sensor 406 is configured to detect the pressure changes and output a signal indicative of the detected pressure to controller 408. Controller 408 is operatively coupled to pressure sensor 406 and memory 410. Controller 408 is configured to receive and process the signal from pressure sensor 406. Memory 410 is configured to store information such as the received signal and an algorithm for signal processing. Controller 408 is optionally operatively coupled to capital unit 404. In an example, nasal cannula 402 is cannula 102 of FIG. 1.

In some implementations, processing the signal includes determining a current breath rate or inspiratory/expiratory ratio of the patient. In some implementations, processing the signal includes identifying peaks in detected pressures over time. In some implementations, processing the signal includes determining an increase or decrease in pressure over time. In some implementations, processing the signal includes identifying an apnea event or a breathing pattern. In some implementations, processing the signal includes applying a filter to the signal to remove noise. In some implementations, processing the signal includes identifying and excluding non-breathing periods (e.g., swallowing, sneezing, talking, or coughing) from the data. By ignoring non-breathing artifacts, controller 408 may more accurately calculate breathing parameters.

In some implementations, the processing the signal involves an average pressure in the signal/data. Maximum peaks, minimum peaks, the average maximum peak over a period of time, and/or the average minimum peak over a period of time may be determined. A pressure threshold may be determined as a percentage (e.g., between about 10% and about 30%) of the average maximum peak. This may allow controller 408 to identify a single breath of the patient based on when the pressure signal follows the following pattern: crosses the average pressure from below the average to above the average, then exceeds the average by at least the pressure threshold, then reaches a maximum peak, then cross the average from above the average to below the average, then reaches a minimum peak, and then crosses the average from below the average to above the average. The above steps may be based on a positive pressure swing, meaning that the pressure change is positive when the patient inhales. Alternatively, the pressure threshold may be determined as a percentage of the average minimum peak, and the algorithm steps are based on a negative pressure swing such that the pressure change is negative when the patient inhales. The pressure sensor used herein can be a relative pressure sensor, measuring the difference in pressure between two ports with one port positioned within the sensing lumen and one outside (measuring pressure of the surrounding environment), so the ports can be swapped to switch between the bases of positive and negative pressure swings.

In some cases, the patient's breathing pattern may change, resulting in breathing that does not reach the threshold for breath detection that has been established from previous average peaks. This can occur if the patient switches to shallow breathing after a period of deep breathing or when a patient switches from closed-mouth to open-mouth breathing. In these cases, an algorithm can detect that there is sufficient pressure change (e.g., one pressure change or multiple pressure changes) to indicate that breathing is occurring even though it is not reaching the minimum threshold of breath detection. The averages are reset and new average peaks are determined from the pressure data.

Any of the above-identified signal processing may be executed by an algorithm stored in memory 410. Data representing information such as pressure signal, current breath rate, inspiratory/expiratory ratio, identified peaks, identified apnea events, or breathing patterns may be stored in memory 410. System 400 may further include a screen or display configured to display any of the above information.

When controller 408 is operatively coupled to capital unit 404, controller 408 may be configured to transmit information or data to capital unit 404. For example, controller 408 is configured to notify capital unit 404 of a non-breathing period or apnea event, and in response capital unit 404 may vary the flow of breathing gas and/or generates an alarm. Controller 408 may transmit a pressure signal, current breath rate, inspiratory/expiratory ratio, identified peaks, identified apnea events, or breathing patterns to capital unit 404. Capital unit 404, in response to receiving any of the above information, may adjust one or more operational parameters. For example, capital unit 404 increases or decreases one or more of breathing gas flow rate, breathing gas humidity, breathing gas temperature, oxygen percentage, oxygen flow rate, or aerosol concentration. This feature may allow a caretaker to wean the user off of respiratory therapy when they demonstrate improved breathing. Capital unit 404, in response to receiving any of the above information, may generate an alarm to notify a user or caretaker. This feature may be beneficial for compliance monitoring.

Capital unit 404 includes a pressurized breathing gas source that may be one of a blower, a compressor, a pressurized tank, a wall air outlet, or any other suitable gas source capable of generating high flow rates (e.g., between about 1 L/min and 60 L/min, or greater). In some implementations, capital unit 404 is configured to output a heated and humidified flow of breathing gas. For example, capital unit 404 contains a vapor transfer unit or hotpot humidifier configured to evaporate water into the flow of breathing gas. In some implementations, capital unit 404 includes a port for oxygen titration. For example, an oxygen tank, oxygen concentrator, or oxygen wall outlet is coupled to capital unit 404, and a flow of oxygen is added to the breathing gas to reach a target oxygen concentration or oxygen flow rate. In some implementations, an aerosolized medicament is entrained in the flow of breathing gas and delivered simultaneously to the patient. For example, a nebulizer is mounted to nasal cannula 402, to capital unit 404, or to a disposable unit on capital unit 404, and the nebulizer emits aerosol medicament particles into the flow of breathing gas.

In some implementations, capital unit 402, nasal cannula 404, pressure sensor 406, controller 408, and memory 410 are parts of one system 400. In other implementations, capital unit 404 is a respiratory system, and a breath sensing device separately houses pressure sensor 406, controller 408, and memory 410, and is adapted to couple to capital unit 404 and nasal cannula 402. The breath sensing device may be packaged and adapted to couple to existing respiratory therapy systems having a suitable pressurized breathing gas source. In some implementations the system 400 includes additional medical monitoring devices such as a pulse oximeter device or transcutaneous carbon dioxide sensor, and the breath sensing data detected by the pressure sensor 406 is displayed at the capital unit 402 with additional parameters detected at the other monitoring devices.

The foregoing is merely illustrative of the principles of the disclosure, and the apparatuses can be practiced by other than the described implementations, which are presented for purposes of illustration and not of limitation. It is to be understood that the apparatuses disclosed herein, while shown for use in high flow therapy systems, may be applied to systems to be used in other ventilation circuits.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

### Illustrative Embodiments

A1. A nasal cannula for breath sensing of a patient being provided high flow respiratory therapy, the nasal cannula comprising:

a cannula body having a first connector configured to receive a first flow of breathing gas and a second connector configured to receive a second flow of breathing gas;

a first nasal prong configured to deliver the first flow of breathing gas to a nare of the patient;

a second nasal prong configured to deliver the second flow of breathing gas to a nare of the patient;

a first sensing lumen having a first sensing tip positioned along the first nasal prong such that, during patient inspiration and expiration, gas flows over the first sensing tip to create a pressure change within the first sensing lumen; and

a first pressure sensor configured to detect a first pressure in the first sensing lumen, the first pressure sensor being positioned at a proximal end of the first sensing lumen opposite the first sensing tip.

A2. The nasal cannula of A1, further comprising:

a second sensing lumen having a second sensing tip positioned along the second nasal prong such that, during patient inspiration and expiration, gas flows over the second sensing tip to create a second pressure change within the second sensing lumen; and

a second pressure sensor configured to detect a second pressure in the second sensing lumen and positioned at a proximal end of the second sensing lumen opposite the second sensing tip.

A3. The nasal cannula of any of A1 or A2, wherein the first sensing lumen comprises:

a housing mounted on the cannula body, and

a tube configured to enable fluid communication between the first sensing tip and the pressure sensor, wherein the tube is in fluid communication with the pressure sensor at the proximal end of the first sensing lumen,

wherein the housing is fluidically connected to the tubing.

A4. The nasal cannula of A3, wherein the tube has a length between about 5 mm and about 3 m.
A5. The nasal cannula of any of A3 or A4, wherein the tube has an internal diameter between about 0.25 mm and about 2.5 mm.
A6. The nasal cannula of any of A3-A5, wherein a control gas flows from a flow generator through at least a portion of the tube in a direction from the proximal end to the sensing tip.
A7. The nasal cannula of A6, wherein the control gas collides with patient breath at a boundary region adjacent to the first sensing tip.
A8. The nasal cannula of A6, wherein the control gas is delivered intermittently as a bolus flow.
A9. The nasal cannula of A6, wherein the control gas has a flow rate between about 0.1 mL/min and about 10 mL/min.
A10. The nasal cannula of any of A6-A9, wherein the flow generator is one of a fan, a syringe pump, a blower, a compressor, a bellows, or a wall air source.

A11. The nasal cannula of any of A6-A10, wherein the nasal cannula is configured with the control gas so as to purge condensation from the first sensing lumen.

A12. The nasal cannula of any of A6-A11, wherein the sensing lumen comprises a tee joint positioned along the tube and configured to convey the control gas into the tube from the flow generator.

A13. The nasal cannula of any of A1-A12, further comprising a temperature sensor configured to measure a breath temperature.
A14. The nasal cannula of any of A1-A13, wherein the detected first pressure indicates a difference between an internal pressure within the first sensing lumen and ambient pressure of the surrounding environment.
A15. The nasal cannula of A14, wherein the difference is at least about 1 Pa to at least about 5 Pa.
A16. The nasal cannula of any of A1-A15, wherein the first pressure sensor is configured to output a pressure signal indicative of the detected first sensing lumen pressure.
A17. The nasal cannula of any of A1-A16, wherein the first sensing tip is angled relative to a longitudinal axis of the first sensing lumen.
A18. The nasal cannula of A17, wherein the first sensing tip comprises a tip face at an angle other than 90 degrees relative to the longitudinal axis.
A19. The nasal cannula of A18, wherein the angle is between about 30 degrees and about 60 degrees.
A20. The nasal cannula of A19, wherein the angle is about 45 degrees.
A21. The nasal cannula of any of A18-A20, wherein the tip face has rounded, beveled, or chamfered edges.
A22. The nasal cannula of any of A1-A21, wherein the sensing tip is positioned on an exterior surface of the first nasal prong.
A23. The nasal cannula of A22, wherein the sensing tip is positioned on a dorsal side of the exterior surface of the first nasal prong.
A24 The nasal cannula of any of A22 or A23, wherein the sensing tip is positioned proximally to a prong tip of the first nasal prong.
A25. The nasal cannula of any of A1-A24, wherein the sensing tip is positioned on the nasal cannula such that the sensing tip resides within the nare of the patient while the cannula is in use.
A26. The nasal cannula of any of A1-A25, wherein the sensing tip has an internal diameter between about 0.25 mm and 2.5 mm.
A27. The nasal cannula of any of A1-A26, wherein the first nasal prong has an internal diameter of about 1.0 to about 4.0 mm.
B1 A breath sensing device configured to monitor patient breathing, the device comprising:

the nasal cannula of any of A1-A27; and

a controller configured to:

receive a pressure signal from the first pressure sensor indicative of the first sensing lumen pressure; and

convert the pressure signal into data.

B2. The breath sensing device of B1, wherein the device is configured to couple to a respiratory therapy system.
B3. The breath sensing device of B2, wherein the controller is configured to send instructions to the respiratory therapy system to change an operating parameter of the respiratory therapy system.
B4. The breath sensing device of B2, wherein the controller is configured to send the data to the respiratory therapy system for determining a change to an operating parameter of the respiratory therapy system.
B5. The breath sensing device of any of B2-B4, wherein the respiratory therapy system is configured to provide the first flow of breathing gas to the nasal cannula.
B6. The breath sensing device of any of B1-B5, wherein the controller is configured to determine periods of patient inspiration and/or expiration based on the data from the first pressure sensor.
B7. The breath sensing device of any of B1-B6, wherein the controller is configured to determine and display at least one of patient expiratory/inspiratory ratio, a current breath rate, or a patient breathing pattern based on the data from the first pressure sensor.
B8. The breath sensing device of any of B1-B7, wherein the controller is configured to apply an algorithm to the data.
B9. The breath sensing device of B8, wherein the algorithm is configured to determine an average pressure in the data.
B10. The breath sensing device of B9, wherein the algorithm is configured to determine maximum and minimum peaks in the data and/or filter the data.
B11. The breath sensing device of B10, wherein the algorithm is configured to determine an average maximum peak over a period of time.
B12. The breath sensing device of B11, wherein the algorithm is configured to determine an average minimum peak over a period of time.
B13. The breath sensing device of B12, wherein the algorithm is configured to determine a pressure threshold as a percentage of the average maximum peak.
B14. The breath sensing device of B13, wherein the algorithm is configured to identify a breath when the pressure:

crosses from below the average pressure to above the average pressure, then
exceeds the average pressure by at least the pressure threshold, then

reaches a maximum peak, then

crosses from above the average pressure to below the average pressure, then

reaches a minimum peak, then
crosses from below the average pressure to above the average pressure.
   B15. The breath sensing device of any of B8-B14, wherein the algorithm is configured to identify and exclude non-breathing periods from the data.
   B16. The breath sensing device of any of B1-B15, comprising the nasal cannula of A2, wherein the controller is configured to average data from the first pressure sensor and the second pressure sensor to generate an average sensing lumen pressure.
   B17. The breath sensing device of B16, wherein the controller is configured to receive data from only one of the first pressure sensor or the second pressure sensor when one of the nares of the patient is blocked.
   C1. A respiratory therapy system comprising:

a capital unit comprising a pressurized gas source;

the nasal cannula of any of A1-A27; and

a delivery tube configured to convey the first flow of breathing gas from the capital unit to the first connector of the nasal cannula.

C2. The respiratory therapy system of C1, further comprising a controller configured to receive a pressure signal from the first pressure sensor indicative of the first sensing lumen pressure and convert the pressure signal to data.
C3. The respiratory therapy system of C2, wherein the controller is configured to change an operating parameter of the capital unit based on the received data.
C4. The respiratory therapy system of any of C2 or C3, wherein the controller is configured to determine periods of patient inspiration and/or expiration based on the data from the first pressure sensor.
C5. The respiratory therapy system of any of C2-C4, wherein the controller is configured to determine and display a patient expiratory/inspiratory ratio, current breath rate, and/or a patient breathing pattern based on the data from the first pressure sensor.
C6. The respiratory therapy system of any of C2-C5, wherein the controller is configured to apply an algorithm to the data.
C7. The respiratory therapy system of C6, wherein the algorithm is configured to determine maximum and minimum peaks in the data and/or filter the data.
C8. The respiratory therapy system of C7, wherein the algorithm is configured to determine an average maximum peak over a period of time.
C9. The respiratory therapy system of any of C7 or C8, wherein the algorithm is configured to determine an average minimum peak over a period of time.
C10. The respiratory therapy system of C9, wherein the algorithm is configured to determine a pressure threshold as a percentage of the average maximum peak.
C11. The respiratory therapy system of C10, wherein the algorithm is configured to identify a breath when the pressure:

crosses from below the average pressure to above the average pressure, then
exceeds the average pressure by at least the pressure threshold, then

reaches a maximum peak, then

crosses from above the average pressure to below the average pressure, then

reaches a minimum peak, then

crosses from below the average pressure to above the average pressure.

C12. The respiratory therapy system of any of C6-C11, wherein the algorithm is configured to identify and exclude non-breathing periods from the data.
C13. The respiratory therapy system of any of C1-C12, comprising the nasal cannula of A3, wherein the controller is configured to average data from the first pressure sensor and the second pressure sensor to generate an average sensing lumen pressure.
C14. The respiratory therapy system of C13, wherein the controller is configured to receive data from only one of the first pressure sensor or the second pressure sensor when one of the nares of the patient is blocked.
C15. The nasal cannula, breath sensing device, or respiratory therapy system of any of A1-A27, B1-B17, or C1-C14, wherein the first flow of breathing gas is heated and humidified.
C16. The nasal cannula, breath sensing device, or respiratory therapy system of C15, wherein the first flow of breathing gas is humidified by a vapor transfer unit or a hotpot humidifier.
C17. The nasal cannula, breath sensing device, or respiratory therapy system of any of A1-A27, B1-B17, or C1-C16, wherein the first flow of breathing gas exits the first nasal prong at an exit velocity of at least about 40 m/s.
C18. The nasal cannula, breath sensing device, or respiratory therapy system of any of A1-A27, B1-B17, or C1-C17, wherein the first flow of breathing gas is delivered at a flow rate between about 1 L/min and 60 L/min.
D1. A method for sensing patient breathing when the patient is provided with high flow respiratory therapy, the method comprising:

delivering a flow of breathing gas to a nare of the patient via a nasal cannula; and

detecting a pressure in a sensing lumen of the nasal cannula using a pressure sensor positioned at a proximal end of the sensing lumen opposite a tip of the sensing lumen,

wherein the sensing lumen is positioned such that, during patient inspiration and expiration, gas flows over the tip of the sensing lumen to create a pressure change within the sensing lumen.

D2. The method of D1, further comprising:

transmitting a signal indicative of the detected pressure from the pressure sensor to a controller.

D3. The method of D2, further comprising:

processing the signal using an algorithm stored in a memory of the controller.

D4. The method of D3, wherein processing the signal comprises determining a current breath rate, and the method further comprises:

displaying the current breath rate.

D5. The method of D4, wherein the signal is indicative of a plurality of pressures detected by the pressure sensor over a period of time, and wherein processing the signal comprises identifying peaks in the plurality of detected pressures over time.
D6. The method of D5, further comprising:

determining an increase or decrease in pressure over time.

D7. The method of any of D5 or D6, further comprising:

identifying an apnea event based on the signal; and

logging the apnea event.

D8. The method of any of D5-D7, further comprising:

determining an average pressure in the data.

D9. The method of D8, further comprising:

determining maximum and minimum peaks in the data and/or filter the data.

D10. The method of D9, further comprising:

determining an average maximum peak over a period of time.

D11. The method of any of D9 or D10, further comprising:

determining an average minimum peak over a period of time.

D12. The method of any of D9-D11, further comprising:

determining a pressure threshold as a percentage of the average maximum peak.

D13. The method of D12, further comprising:

determining a breath when the pressure:
crosses from below the average pressure to above the average pressure, then exceeds the average pressure by at least the pressure threshold reaches a maximum peak, then
crosses from above the average pressure to below the average pressure, then reaches a minimum peak, then
crosses from below the average pressure to above the average pressure.
   D14. The method of any of D4-D13, further comprising:

operating the controller to alter an operating parameter of the respiratory therapy based on the detected pressure.

D15. The method of D14, wherein the operating parameter is one of a breathing gas flow rate, an oxygen percentage of the flow of breathing gas, a breathing gas temperature, a breathing gas humidity, or an aerosolized medicament flow rate.
D16. The method of any of D1-D15, wherein the detected pressure is a differential pressure between a pressure within the sensing lumen and an ambient pressure.
D17. The method of any of D1-D16, further comprising:
conveying a control flow of gas through the sensing lumen from a flow generator.
D18. The method of D17, further comprising:
purging condensation from the sensing lumen with the control flow of gas.
D19. The method of any of D1-D18, further comprising:

humidifying the flow of breathing gas using a vapor transfer unit or a hotpot humidifier.

E1. A method of manufacturing a nasal cannula for respiratory therapy and breath sensing, the method comprising:

maintaining a first mandrel, a second mandrel, and a third mandrel in a fixed arrangement with respect to each other, wherein the third mandrel is positioned along at least a portion of the first mandrel at a distance from the first mandrel;

coating the arrangement with a material;

curing the coated arrangement;

trimming at least one coated mandrel to create an opening in the coating of the trimmed mandrel; and

removing the cured coating from the arrangement,

wherein the coating on the first, second, and third mandrels form a first nasal prong, a second nasal prong, and a sensing lumen, respectively.

E2. The method of E1, wherein the step of coating comprises:

immersing the arrangement in the material; and

removing the arrangement from the material.

E3. The method of E1, wherein the step of coating comprises:

spraying the material onto the arrangement.

E4. The method of E1, wherein the first, second, and third mandrels are fixedly held on a substrate.

1. A nasal cannula for breath sensing of a patient being provided high flow respiratory therapy, the nasal cannula comprising:
a cannula body having a first connector configured to receive a first flow of breathing gas and a second connector configured to receive a second flow of breathing gas;
a first nasal prong configured to deliver the first flow of breathing gas to a nare of the patient;
a second nasal prong configured to deliver the second flow of breathing gas to a nare of the patient;
a first sensing lumen having a first sensing tip positioned along the first nasal prong such that, during patient inspiration and expiration, gas flows over the first sensing tip to create a pressure change within the first sensing lumen; and
a first pressure sensor configured to detect a first pressure in the first sensing lumen, the first pressure sensor being positioned at a proximal end of the first sensing lumen opposite the first sensing tip.

2. The nasal cannula of claim 1, further comprising:
a second sensing lumen having a second sensing tip positioned along the second nasal prong such that, during patient inspiration and expiration, gas flows over the second sensing tip to create a second pressure change within the second sensing lumen; and
a second pressure sensor configured to detect a second pressure in the second sensing lumen and positioned at a proximal end of the second sensing lumen opposite the second sensing tip.

3. The nasal cannula of claim 1, wherein the first sensing lumen comprises:
a housing mounted on the cannula body, and
a tube configured to enable fluid communication between the first sensing tip and the pressure sensor, wherein the tube is in fluid communication with the pressure sensor at the proximal end of the first sensing lumen,
wherein the housing is fluidically connected to the tubing.

4. The nasal cannula of claim 3, wherein the tube has a length between about 5 mm and about 3 m.

5. The nasal cannula of claim 3, wherein the tube has an internal diameter between about 0.25 mm and about 2.5 mm.

6. The nasal cannula of claim 3, wherein a control gas flows from a flow generator through at least a portion of the tube in a direction from the proximal end to the sensing tip.

7. The nasal cannula of claim 6, wherein the control gas collides with patient breath at a boundary region adjacent to the first sensing tip.

8. The nasal cannula of claim 6, wherein the control gas is delivered intermittently as a bolus flow.

9. The nasal cannula of claim 6, wherein the control gas has a flow rate between about 0.1 mL/min and about 10 mL/min.

10. The nasal cannula of claim 6, wherein the flow generator is one of a fan, a syringe pump, a blower, a compressor, a bellows, or a wall air source.

11. The nasal cannula of claim 6, wherein the nasal cannula is configured with the control gas so as to purge condensation from the first sensing lumen.

12. The nasal cannula of claim 6, wherein the sensing lumen comprises a tee joint positioned along the tube and configured to convey the control gas into the tube from the flow generator.

13. The nasal cannula of claim 1, further comprising a temperature sensor configured to measure a breath temperature.

14. The nasal cannula of claim 1, wherein the detected first pressure indicates a difference between an internal pressure within the first sensing lumen and ambient pressure of the surrounding environment.

15. The nasal cannula of claim 14, wherein the difference is at least about 1 Pa to at least about 5 Pa.

16. The nasal cannula of claim 1, wherein the first pressure sensor is configured to output a pressure signal indicative of the detected first sensing lumen pressure.

17. The nasal cannula of claim 1, wherein the first sensing tip is angled relative to a longitudinal axis of the first sensing lumen.

18. The nasal cannula of claim 17, wherein the first sensing tip comprises a tip face at an angle other than 90 degrees relative to the longitudinal axis.

19. The nasal cannula of claim 18, wherein the angle is between about 30 degrees and about 60 degrees.

20. The nasal cannula of claim 19, wherein the angle is about 45 degrees.

21. The nasal cannula of claim 18, wherein the tip face has rounded, beveled, or chamfered edges.

22. The nasal cannula of claim 1, wherein the sensing tip is positioned on an exterior surface of the first nasal prong.

23. The nasal cannula of claim 22, wherein the sensing tip is positioned on a dorsal side of the exterior surface of the first nasal prong.

24. The nasal cannula of claim 22, wherein the sensing tip is positioned proximally to a prong tip of the first nasal prong.

25. The nasal cannula of claim 1, wherein the sensing tip is positioned on the nasal cannula such that the sensing tip resides within the nare of the patient while the cannula is in use.

26. The nasal cannula of claim 1, wherein the sensing tip has an internal diameter between about 0.25 mm and 2.5 mm.

27. The nasal cannula of claim 1, wherein the first nasal prong has an internal diameter of about 1.0 to about 4.0 mm.

28-85. (canceled)
